(19) United States
(12) Patent Application Publication
(10) Pub. No.: US 2026/0224797 A1
(43) Pub. Date: Aug. 6, 2026

(54) System And Method For Negative Pressure Wound Therapy

(71) Applicant: Solventum Intellectual Properties Company, Maplewood, MN (US)

(72) Inventors: Benjamin A. PRATT, Poole (GB); Robert HOWARD, Westmeath (GB); Kevin H. DAI, Dugan, NC (US)

(21) Appl. No.: 19/147,172

(22) PCT Filed: Jan. 9, 2024
(86) PCT No.: PCT/IB2024/050192

§ 371 Date: Jul. 10, 2025

Related U.S. Application Data
(60) Provisional application No. 63/438,052, filed on Jan. 10, 2023.

(51) Int. Cl. A61M 1/00

(52) U.S. Cl. CPC A61M 1/966; A61M 1/91; A61M 2205/3344

(57) ABSTRACT

A system for negative pressure wound therapy includes a controller configured to operate a pump in a plurality of cycles based at least on a negative wound pressure generated by the pump at a wound site. The controller is further configured to define an upper negative target pressure, a lower negative target pressure, a fall line, and a rise line. The controller is further configured to switch off the pump when the negative wound pressure reaches the upper negative target pressure of the cycle. The controller is further configured to switch on the pump when the negative wound pressure decreases from the upper negative target pressure to the rise line of the cycle due to a leakage from the wound site after switching off the pump. The controller is further configured to operate the pump such that the negative wound pressure increases along the rise line of the cycle.

### CROSS-REFERENCE TO RELATED APPLICATIONS

This application is a U.S. National Stage Entry of PCT International Application No. PCT/IB 2024/050192, filed on Jan. 9, 2024, which claims the benefit of priority to U.S. Provisional Application No. 63/438,052, filed on Jan. 10, 2023, which is incorporated herein by reference in its entirety.

### TECHNICAL FIELD

The present disclosure relates generally to a system and a method for negative pressure wound therapy.

### BACKGROUND

Negative pressure wound therapy (NPWT) systems are embodied as sealed wound-care systems particularly indicated for chronic persistent wounds and/or complicated wounds. Specifically, NPWT systems involve application of a pressure that is reduced relative to the surroundings (commonly referred to as “negative pressure”) to the wound for promoting wound healing. The provision of NPWT over a therapeutic life cycle is increasingly being performed with small portable therapy devices that are powered by batteries rather than a connection to an electrical outlet. Some therapies involve negative pressures that vary between a maximum and a minimum pressure value that may be set as an input by a user as a range of negative pressures desired for the therapy.

The small portable therapy devices are often disposable, and typically use non-rechargeable batteries. Limiting factors for such devices include the ability of their pumps to run harder in case of a leakage through the device, e.g., through a dressing covering the wound due to an imperfect seal. The leakage may negatively impact an operational life of the batteries as the pump may need to offset the leakage to provide the desired therapy. A user experience may be negatively impacted if the user needs to replace the batteries too frequently.

### SUMMARY

Generally, the present disclosure relates to a system and a method for negative pressure wound therapy.

In a first aspect, the present disclosure provides a system for negative pressure wound therapy. The system includes a pump fluidly coupled to a wound site and configured to generate a negative wound pressure at the wound site. The system further includes a pressure sensor disposed in fluid communication with the wound site and configured to generate a pressure signal indicative of the negative wound pressure at the wound site. The system further includes a controller communicably coupled to the pump and the pressure sensor. The controller is configured to determine the negative wound pressure based on the pressure signal received from the pressure sensor and operate the pump in a plurality of cycles based at least on the negative wound pressure. The plurality of cycles are disposed adjacent to each other with respect to time. In each cycle, the controller is further configured to: define an upper negative target pressure and a lower negative target pressure, a magnitude of the upper negative target pressure being greater than a magnitude of the lower negative target pressure; determine a fall line decreasing from the upper negative target pressure to the lower negative target pressure; determine a rise line increasing from the lower negative target pressure to the upper negative target pressure and extending from the fall line, the fall line of a subsequent cycle of the plurality of cycles extending from the rise line of the cycle; switch off the pump when the negative wound pressure reaches the upper negative target pressure of the cycle; switch on the pump when the negative wound pressure decreases from the upper negative target pressure to the rise line of the cycle above the lower negative target pressure due to a leakage from the wound site after switching off the pump; and operate the pump such that the negative wound pressure increases along the rise line of the cycle.

In a second aspect, the present disclosure provides a system for negative pressure wound therapy. The system includes a pump fluidly coupled to a wound site and configured to generate a negative wound pressure at the wound site. The system further includes a pressure sensor disposed in fluid communication with the wound site and configured to generate a pressure signal indicative of the negative wound pressure at the wound site. The system further includes a controller communicably coupled to the pump and the pressure sensor. The controller is configured to determine the negative wound pressure based on the pressure signal received from the pressure sensor and operate the pump in a plurality of cycles. The plurality of cycles are disposed adjacent to each other with respect to time. In each cycle, the controller is further configured to: define an upper negative target pressure and a lower negative target pressure, a magnitude of the upper negative target pressure being greater than a magnitude of the lower negative target pressure; switch off the pump when the negative wound pressure reaches the upper negative target pressure of the cycle; determine a rate of decrease of the negative wound pressure with respect to time from the upper negative target pressure to the lower negative target pressure of the cycle due to a leakage from the wound site after switching off the pump; determine a rate of increase of the negative wound pressure with respect to time having a same magnitude as that of the rate of decrease of the negative wound pressure; switch on the pump when the negative wound pressure reaches the lower negative target pressure of the cycle; and operate the pump to increase the negative wound pressure from the lower negative target pressure at the rate of increase of the negative wound pressure.

In a third aspect, the present disclosure provides a method for negative pressure wound therapy. The method includes providing a pump fluidly coupled to a wound site and configured to generate a negative wound pressure at the wound site. The method further includes providing a pressure sensor disposed in fluid communication with the wound site and configured to generate a pressure signal indicative of the negative wound pressure at the wound site. The method further includes determining, via a controller, the negative wound pressure based on the pressure signal received from the pressure sensor. The method further includes operating, via the controller, the pump in a plurality of cycles based at least on the negative wound pressure. The plurality of cycles are disposed adjacent to each other with respect to time. Each cycle includes: defining an upper negative target pressure and a lower negative target pressure, a magnitude of the upper negative target pressure being greater than a magnitude of the lower negative target pressure; determining a fall line decreasing from the upper negative target pressure to the lower negative target pressure; determining a rise line increasing from the lower negative target pressure to the upper negative target pressure and extending from the fall line, the fall line of a subsequent cycle of the plurality of cycles extending from the rise line of the cycle; switching off the pump when the negative wound pressure reaches the upper negative target pressure of the cycle; switching on the pump when the negative wound pressure decreases from the upper negative target pressure to the rise line of the cycle above the lower negative target pressure due to a leakage from the wound site after switching off the pump; and operating the pump such that the negative wound pressure increases along the rise line of the cycle.

In a fourth aspect, the present disclosure provides a method for negative pressure wound therapy. The method includes providing a pump fluidly coupled to a wound site and configured to generate a negative wound pressure at the wound site. The method further includes providing a pressure sensor disposed in fluid communication with the wound site and configured to generate a pressure signal indicative of the negative wound pressure at the wound site. The method further includes determining, via a controller, the negative wound pressure based on the pressure signal received from the pressure sensor. The method further includes operating, via the controller, the pump in a plurality of cycles based at least on the negative wound pressure. The plurality of cycles are disposed adjacent to each other with respect to time. Each cycle includes: defining an upper negative target pressure and a lower negative target pressure, a magnitude of the upper negative target pressure being greater than a magnitude of the lower negative target pressure; switching off the pump when the negative wound pressure reaches the upper negative target pressure of the cycle; determining a rate of decrease of the negative wound pressure with respect to time from the upper negative target pressure to the lower negative target pressure of the cycle due to a leakage from the wound site after switching off the pump; determining a rate of increase of the negative wound pressure with respect to time having a same magnitude as that of the rate of decrease of the negative wound pressure; switching on the pump when the negative wound pressure reaches the lower negative target pressure of the cycle; and operating the pump to increase the negative wound pressure from the lower negative target pressure at the rate of increase of the negative wound pressure.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 illustrates a schematic view of a system for negative pressure wound therapy of a wound site, according to an embodiment of the present disclosure;

FIG. 2 is a graph illustrating change in a negative wound pressure at the wound site with respect to time due to a leakage condition, according to an embodiment of the present disclosure;

FIG. 3 is a graph illustrating change in the negative wound pressure at the wound site with respect to time due to the leakage condition, according to another embodiment of the present disclosure;

FIG. 4 is a graph illustrating change in the negative wound pressure at the wound site with respect to time due to the leakage condition, according to another embodiment of the present disclosure;

FIG. 5 is a graph illustrating change in the negative wound pressure at the wound site with respect to time due to the leakage condition, according to another embodiment of the present disclosure;

FIG. 6 is a graph illustrating change in the negative wound pressure at the wound site with respect to time due to the leakage condition, according to another embodiment of the present disclosure;

FIG. 7 is a graph illustrating change in the negative wound pressure at the wound site with respect to time due to the leakage condition, according to another embodiment of the present disclosure;

FIG. 8 is a flow chart illustrating a method for negative pressure wound therapy, according to an embodiment of the present disclosure; and

FIG. 9 is a flow chart illustrating a method for negative pressure wound therapy, according to another embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As recited herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20 % for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

Terms such as same, equal, uniform, constant, strictly, and the like, are understood to be within the usual tolerances or measuring error applicable to the particular circumstance rather than requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, “at least one of A and B” should be understood to mean “only A, only B, or both A and B”.

Unless specified or limited otherwise, the terms “attached,” “connected,” “coupled”, and variations thereof, are used broadly and encompass both direct physical connections, or indirect physical connections between two or more components that are connected together by one or more additional components. For example, a first component may be coupled to a second component by being directly connected together or by being connected by a third component. In some examples, coupling, connection, and attachment may also include mechanical, thermal, electrical, or chemical coupling (such as a chemical bond) in some contexts.

As used herein, the terms “layer,” “sheet,” and “dressing,” or variations thereof, are used to describe an article having a thickness that is small relative to its length and width.

As used herein, the term “negative pressure” generally refers to a pressure lower than a local ambient pressure in a local environment outside of a sealed treatment environment provided by a dressing at a wound site. In many cases, the local ambient pressure may also be the atmospheric pressure at which the wound site is located. Alternatively, the pressure may be less than a hydrostatic pressure associated with a tissue at the wound site. A reference to an increase in the negative pressure typically refers to a decrease in an absolute pressure, while a decrease in the negative pressure typically refers to an increase in the absolute pressure.

As used herein, the term “wound” may include, for example, chronic, acute, traumatic, subacute, closed surgical wounds, or dehiscence wounds, partially thick burns, ulcers (such as, diabetic, compressive, or venous insufficiency ulcers), flaps, and grafts. The term may also include an open abdomen area of a patient.

As used herein, the term “wound site” may include a tissue site, such as, bone tissue, adipose tissue, muscle tissue, nerve tissue, skin tissue, vascular tissue, connective tissue, cartilage, tendons, or ligaments. The term “wound site” may also refer to an area of a tissue that is not necessarily a wound or a defect but may be desired to add or promote additional tissue growth. For example, negative pressure therapy can be used at a particular tissue area to grow additional tissue that may be harvested or transplanted to another tissue site. The wound site may also include an area wherein a surgical incision has been previously performed.

Negative pressure wound therapies are increasingly being performed with small portable therapy devices that are powered by batteries rather than a connection to an electrical outlet. Such small portable therapy devices are often disposable, and typically use non-rechargeable batteries. Limiting factors for such devices include the ability of their pumps to run harder in case of a leakage through the device, e.g., through a dressing covering the wound due to an imperfect seal. The leakage may negatively impact an operational life of the batteries as the pump may need to offset the leakage to provide the desired therapy. A user experience may be negatively impacted if the user needs to replace the batteries too frequently.

The present disclosure provides a system for negative pressure wound therapy. The system includes a pump fluidly coupled to a wound site and configured to generate a negative wound pressure at the wound site. The system further includes a pressure sensor disposed in fluid communication with the wound site and configured to generate a pressure signal indicative of the negative wound pressure at the wound site. The system further includes a controller communicably coupled to the pump and the pressure sensor. The controller is configured to determine the negative wound pressure based on the pressure signal received from the pressure sensor and operate the pump in a plurality of cycles based at least on the negative wound pressure. The plurality of cycles are disposed adjacent to each other with respect to time. In each cycle, the controller is further configured to: define an upper negative target pressure and a lower negative target pressure, a magnitude of the upper negative target pressure being greater than a magnitude of the lower negative target pressure; determine a fall line decreasing from the upper negative target pressure to the lower negative target pressure; determine a rise line increasing from the lower negative target pressure to the upper negative target pressure and extending from the fall line, the fall line of a subsequent cycle of the plurality of cycles extending from the rise line of the cycle; switch off the pump when the negative wound pressure reaches the upper negative target pressure of the cycle; switch on the pump when the negative wound pressure decreases from the upper negative target pressure to the rise line of the cycle above the lower negative target pressure due to a leakage from the wound site after switching off the pump; operate the pump such that the negative wound pressure increases along the rise line of the cycle.

The system of the present disclosure may control an application of the negative wound pressure at the wound site in a manner that is not only therapeutically effective, but also allows for extending an operational life of batteries in case of leakage from the wound site. As the controller switches off the pump when the negative wound pressure reaches the upper negative target pressure of the cycle, the negative wound pressure may decrease from the upper negative target pressure due to the leakage from the wound site. Subsequently, the controller may switch on the pump when the negative wound pressure decreases to the rise line of the cycle to operate the pump such that the negative wound pressure increases along the rise line of the cycle.

Thus, the system may conserve energy by not allowing the negative wound pressure to unnecessarily reduce to the lower negative target pressure and then execute a corresponding pressure rise, i.e., driving the pump from the lower negative target pressure to the upper negative target pressure. Instead, the controller may switch on the pump when the negative wound pressure decreases to the rise line of the cycle, thereby reducing a power required by the pump to drive the negative wound pressure to the upper negative target pressure. An average negative wound pressure applied at the wound site may not be significantly reduced due to the proposed control strategy, thereby maintaining the desired therapeutic effects of the negative wound pressure at the wound site.

The system may be advantageously applied to small portable therapy devices that typically run on batteries instead of a connection to an electrical outlet. Such devices may not include any purge valves that essentially drive the negative wound pressure from the upper negative target pressure to the lower negative target pressure. In such devices, the system may apply continuous negative wound pressure in the presence of no or very low leakage, and use the aforementioned control strategy if leakage occurs as the leakage reduces the negative wound pressure from the upper negative target pressure. This may extend the operational life of the batteries and enhance a user experience by not having to replace the batteries too frequently due to the leakage.

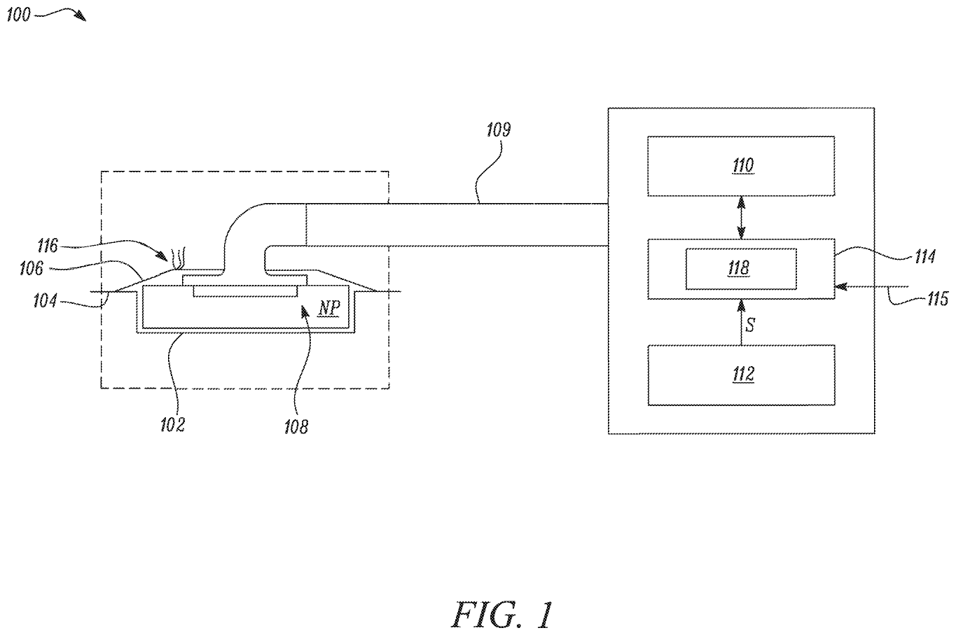

Referring now to Figures, FIG. 1 illustrates a schematic view of a system 100 for negative pressure wound therapy of a wound site 102. The system 100 may be used for healing the wound site 102 of a user (not shown). A skin 104 of the user includes the wound site 102 that is to be treated. In some examples, the wound site 102 may be a bodily tissue of any human, animal, or other organism. While wound site 102 may include a wound, diseased tissue, or defective tissue, the wound site 102 may also include a healthy tissue that is not wounded, diseased, or defective. The application of negative pressure wound therapy to the wound site 102 may be used to promote drainage of wound exudates and other liquids from the wound site 102, as well as promote the growth of additional tissue. The application of negative pressure wound therapy to non-wounded or non-defective tissue, including healthy tissue, may be used to promote the growth of additional tissue that may be harvested and transplanted to another tissue location.

The system 100 includes a wound dressing 106 covering the wound site 102 and defining a sealed volume 108 around the wound site 102. In some examples, the wound dressing 106 may be adapted to seal the wound site 102 and create a therapeutic environment proximal to the wound site 102 for maintaining a negative wound pressure at the wound site 102. In some examples, the wound dressing 106 is preferably detachable, and may be disposable, reusable, or recyclable.

It should be understood that the wound dressing 106 is schematically shown for the purpose of illustration, and the wound dressing 106 may include components, such as a drape or cover, a tissue interface, etc. In some examples, the tissue interface may partially or completely fill the wound site 102 or may be placed over the wound site 102. For example, the tissue interface may include a manifold (not shown) which may be placed adjacent to or in contact with the wound site 102. The manifold may be a biocompatible, porous material that is capable of being placed in contact with wound site 102 and distributing a negative wound pressure to the wound site 102. For example, the manifold may be made from foam, gauze, felted mat, or any other material suitable for biological applications. The manifold may include a plurality of flow channels or pathways to facilitate distribution of the negative wound pressure or fluids to or from the wound site 102. In some examples, the manifold may be secured to the wound site 102 using the drape or cover. In some examples, the drape may provide a bacterial barrier and protection from physical trauma.

The system 100 further includes a pump 110 fluidly coupled to the wound site 102 and configured to generate a negative wound pressure NP at the wound site 102. In some examples, the pump 110 is configured to generate the negative wound pressure NP within the sealed volume 108. In some examples, the pump 110 is fluidly coupled to the wound site 102 (or the wound dressing 106) via a conduit 109 for generating the negative wound pressure NP at the wound site 102. The term “conduit”, as used herein, broadly refers to a tube, a pipe, a hose, a conduit, or other structure with one or more lumina adapted to convey one or more fluids between two ends.

Fluid mechanics of using a pump to reduce pressure in another component or location, such as within a sealed therapeutic environment, can be mathematically complex. However, the basic principles of fluid mechanics applicable to the negative pressure wound therapy are generally well-known to those skilled in the art, and the process of reducing pressure may be described illustratively herein as “delivering,” “distributing,”, “generating”, “applying”, or “achieving” the negative wound pressure, for example.

In some examples, the pump 110 may be any type of manually, mechanically, or electrically operated pump. Non-limiting example of the pump 110 may include devices that are driven by stored energy, and which are capable of reducing pressure in a sealed volume. Examples of such devices include, without limitation, pumps driven by primary and secondary cells, piezoelectric energy, spring energy, solar energy, kinetic energy, energy stored in capacitors, combustion, and energy developed by Sterling or similar cycles. In some examples, the pump 110 may include a vacuum pump, a suction pump, or a wall suction port, available at many healthcare facilities, or a micro-pump, for example. The pump 110 may further include other components, such as, sensors, processing units, alarm indicators, memories, databases, software, display devices, and/or user interfaces that may further facilitate the negative pressure wound therapy.

In some examples, the system 100 may also be used to provide instillation of topical treatment solutions and debridement to the wound site 102 for therapeutic purposes. In some examples, the system 100 may further include features involving aspirating the wound site 102 together with provision of additional fluids for irrigating and/or cleansing the wound site 102. Thereafter, wound fluids including both wound exudates and the irrigation/cleansing fluids are drawn off by the pump 110, which may be collected in an exudate container (not shown). In some examples, the negative wound pressure NP applied at the wound site 102 may induce macrostrain and microstrain at the wound site 102, as well as remove the wound fluids from the wound site 102. In some examples, the conduit 109 may be additionally used to collect the wound fluids from the wound site 102.

The system 100 further includes a pressure sensor 112 disposed in fluid communication with the wound site 102 and configured to generate a pressure signal S indicative of the negative wound pressure NP at the wound site 102. In some examples, the pressure sensor 112 is in fluid communication with the sealed volume 108. In one non-limiting example, the pressure sensor 112 may be a silicon piezo-resistive gauge pressure sensor. In some examples, the pressure sensor 112 may determine the negative wound pressure NP at the wound site 102 via the one or more lumens of the conduit 109. The system 100 further includes a controller 114 communicably coupled to the pump 110 and the pressure sensor 112. In some examples, the pump 110 may be driven by an electric motor (not shown) communicably coupled to the controller 114. In some examples, the controller 114 is configured to determine the negative wound pressure NP based on the pressure signal S received from the pressure sensor 112.

In some examples, the controller 114 may be embodied in a number of different ways. For example, the controller 114 may be embodied as various processing means, such as one or more of a microprocessor, or other processing elements, a coprocessor, or various other computing or processing devices, including integrated circuits, such as, for example, an ASIC (application specific integrated circuit), an FPGA (field programmable gate array), or the like. In some examples, the controller 114 may be configured to execute instructions stored in a memory 118. In some examples, the memory 118 may be a cache memory, a system memory, or any other memory.

As such, whether configured by hardware, or by a combination of hardware and software, the controller 114 may represent an entity (e.g., physically embodied in a circuitry-in the form of a processing circuitry) capable of performing operations according to some embodiments while configured accordingly. Thus, for example, when the controller 114 is embodied as an ASIC, FPGA, or the like, the controller 114 may have specifically configured hardware for conducting the operations described herein. Alternatively, as another example, when the controller 114 may be embodied as an executor of software instructions, the instructions may specifically configure the controller 114 to perform the operations described herein.

In some examples, the memory 118 may be a main memory, a static memory, or a dynamic memory. The memory 118 may include, but may not limited to, computer readable storage media, such as various types of volatile and non-volatile storage media, including, but not limited to, random access memory (RAM), read-only memory (ROM), programmable read-only memory, electrically programmable read-only memory (EPROM), electrically erasable programmable read-only memory (EEPROM), flash memory, magnetic tape or disk, optical media, solid-state memory array, and/or the like.

In some examples, the system 100 may be a part of a small portable therapy device driven by a rechargeable or a non-rechargeable power source, e.g., batteries. Preferably, the pump 110 utilizes low amounts of power and is capable of operating for an extended period of time on a single charge of a battery (not shown), such as, for example, a diaphragm pump, a piezoelectric pump, etc. In some examples, the diaphragm pump may be designed to pump liquids, slurries, sludge, or a combination thereof. For example, the diaphragm pump may be designed to handle liquids with different viscosities and even liquids that contain a considerable amount of solid material.

In some examples, the electric motor powering the pump 110 may be a direct-current (DC) motor powered by a direct current (DC) power supply, such as, for example, the battery. The applied power may be varied to control a speed of the electric motor (e.g., by varying either a current or a voltage applied to the electric motor), thereby controlling the negative wound pressure NP generated at the wound site 102.

In some examples, the controller 114 may receive an input 115 from a clinician/user for setting a desired target pressure to be delivered to the wound site 102. In some examples, the target pressure may be a fixed pressure value determined by the clinician/user for the negative pressure wound therapy at the wound site 105 and then provided as the input 115 to the controller 114. The target pressure may vary based on the wound site 102 but may generally be chosen based on a type of tissue making up the wound site 102, a type of injury or wound (if any), a medical condition of the user, and a preference of the clinician. After receiving the target pressure, the controller 114 may control the pump 110 to generate the target pressure at the wound site 102.

In some examples, during the negative pressure wound therapy, the controller 114 may control the pump 110 to achieve the desired target pressure and then deactivate the pump 110. In some examples, the system 100 may not include any provision for venting the wound site 102 to the atmosphere. Thus, in theory, if the system 100 has no leak, the negative wound pressure NP may remain at the desired target pressure set by the clinician/user. However, due to a leakage 116 in the system 100 (e.g., due to imperfect sealing of the wound dressing 106), the negative wound pressure NP may reduce from the desired target pressure as the pump 110 is deactivated upon attaining the desired target pressure. In some examples, the leakage 116 occurs from the sealed volume 108. In some examples, the controller 114 may identify presence of the leakage 116 in the wound dressing 106 based on the pressure signal S received from the pressure sensor 112. For example, the controller 114 may monitor the negative wound pressure NP to determine presence of the leakage 116.

Alternatively, the applied voltage provided to the electric motor for driving the pump 110 may be used to determine the presence of the leakage 116. For examples, the applied voltage may be used to control a pump pressure, and ultimately achieve the desired target pressure at the wound site 102. Correspondingly, the applied voltage provides an indication of the pump pressure and may be monitored by the controller 114, which in turn may determine a time rate of change of the applied voltage that necessarily corresponds to a time rate of change of the pump pressure. The controller 114 may use this computation for determining a flow rate of air between the pump 110 and the wound site 102. The flow rate may indicate an extent to which the wound dressing 106 or other components of the system 100 may be leaking (e.g., through the leakage 116) to reduce the negative wound pressure NP at the wound site 102 below the desired target pressure for the negative pressure wound therapy. For example, a high flow rate may indicate that the wound dressing 106 is in a “high leakage condition”, while a lower flow rate may indicate that the wound dressing 106 is in a “low leakage condition”. The leakage condition may require the pump 110 to run intermittently or continuously in order to offset the leakage 116. In some examples, the controller 114 may also determine the leakage condition based on the pressure signal S received from the pressure sensor 112.

In some examples, the provision of negative pressure wound therapy over a therapeutic life cycle, which may be a 7-day or more therapeutic life cycle, may be performed by a small portable therapy device powered by a primary cell battery (e.g., a non-rechargeable lithium battery). In some examples, the system 100 may be a part of the small portable therapy device. Alternatively, the small portable therapy device may be powered by a secondary cell battery, which may be a rechargeable lithium-ion battery pack or a rechargeable lithium-polymer battery pack. A time period of operation of the primary cell battery or a time period between subsequent recharges of the secondary cell battery may be increased as a result of the system and methods disclosed herein.

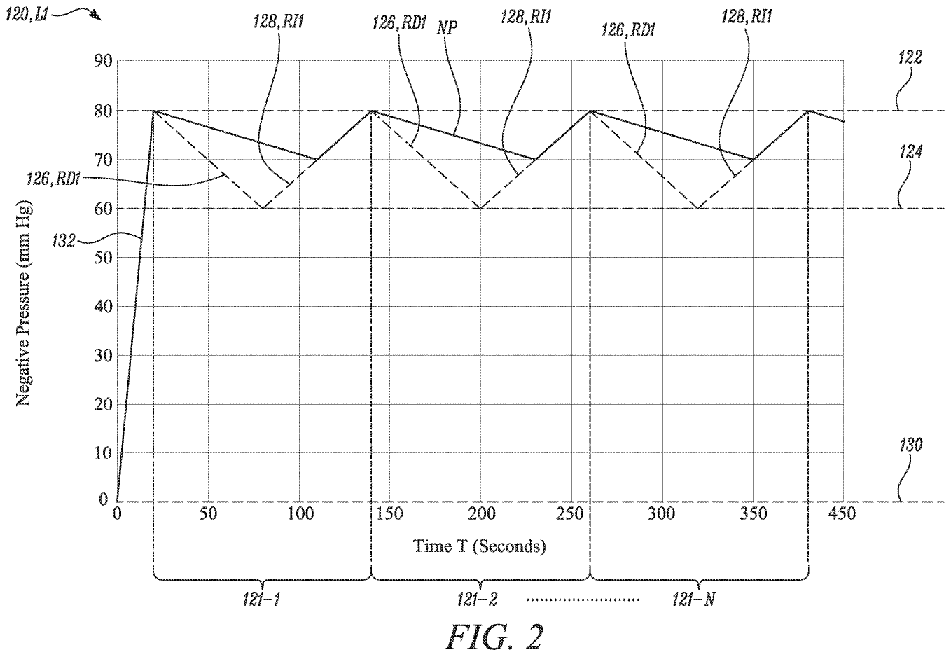

FIG. 2 is a graph 120 illustrating change in the negative wound pressure NP at the wound site 102 (shown in FIG. 1) with respect to time T. In some examples, the negative wound pressure NP is measured in millimeter of mercury (mm Hg) and time T in seconds(s), however, any suitable units may be chosen based on application requirements and a type of the pressure sensor 112 (shown in FIG. 1). Magnitude of the negative wound pressure NP (in mm Hg) is shown along the vertical axis or ordinate of the graph 120 and magnitude of time T (in seconds) is shown along the horizontal axis or abscissa.

Referring to FIGS. 1 and 2, the controller 114 is further configured to operate the pump 110 in a plurality of cycles 121-1, 121-2, . . . , 121-N (collectively, cycles 121) based at least on the negative wound pressure NP, wherein N is a positive integer corresponding to a total number of cycles 121 (e.g., N=10, 20, etc.). In some examples, the plurality of cycles 121 are disposed adjacent to each other with respect to time T. In some examples, the graph 120 may correspond to a leakage condition L1 of the leakage 116 in the system 100. In some examples, the leakage condition L1 may represent a leakage flow of 30 cubic centimeters per minute (cc/min). However, it should be understood that the magnitude of the leakage flow is purely exemplary, and the actual value may vary based on application.

In each cycle 121, the controller 114 is further configured to define an upper negative target pressure 122 and a lower negative target pressure 124. A magnitude of the upper negative target pressure 122 is greater than a magnitude of the lower negative target pressure 124. In some examples, the upper negative target pressure 122 and the lower negative target pressure 124 may be provided as an input to the controller 114 by the clinician/user. Alternatively, only the upper negative target pressure 122 is received as the input and the lower negative target pressure 124 may be preset and stored within the memory 118.

In the illustrated example of FIG. 2, the upper negative target pressure 122 is 80 mm Hg and the lower negative target pressure 124 is 60 mm Hg. However, it should be understood that the magnitude of the upper negative target pressure 122 and the lower negative target pressure 124 may vary based on application requirements. In the illustrated embodiment of FIG. 2, the upper negative target pressure 122 is constant across the plurality of cycles 121. Further, the lower negative target pressure 124 is constant across the plurality of cycles 121.

In each cycle 121, the controller 114 is further configured to determine a fall line 126 decreasing from the upper negative target pressure 122 to the lower negative target pressure 124. In some examples, the fall line 126 may be a straight line or curvilinear. In the illustrated embodiment of FIG. 2, the fall line 126 is linear. The fall line 126 is shown dotted for illustrative purposes as the fall line 126 represents a portion of the cycle 121 that is running in the background. The actual variation of the negative wound pressure NP with respect to time T is represented by a solid line.

In some examples, in each cycle 121, the controller 114 is further configured to define a rate of decrease RD1 of the negative wound pressure NP with respect to time T from the upper negative target pressure 122 to the lower negative target pressure 124. In some examples, in each cycle 121, the controller 114 is further configured to determine the fall line 126 based on the rate of decrease RD1 of the negative wound pressure NP, such that the fall line 126 is linear. In some examples, the rate of decrease RD1 of the negative wound pressure NP with respect to time T may be received as an input from the clinician/user or may be defined based on a type of the negative pressure wound therapy required. In some examples, the controller 114 is further configured to store the rate of decrease RD1 of the negative wound pressure NP with respect to time T in the memory 118. It should be understood that the rate of decrease RD1 of the negative wound pressure NP may be suitably adjusted based on application requirements.

In each cycle 121, the controller 114 is further configured to determine a rise line 128 increasing from the lower negative target pressure 124 to the upper negative target pressure 122 and extending from the fall line 126. In some examples, the rise line 128 may be a straight line or curvilinear. In the illustrated embodiment of FIG. 2, the rise line 128 is linear. In some examples, in each cycle 121, the controller 114 is further configured to define a rate of increase RI1 of the negative wound pressure NP with respect to time T from the lower negative target pressure 124 to the upper negative target pressure 122. In some examples, in each cycle 121, the controller 114 is further configured to determine the rise line 128 based on the rate of increase RI1 of the negative wound pressure NP, such that the rise line 128 is linear.

In some examples, the rate of increase RI1 of the negative wound pressure NP with respect to time T may be received as an input from the clinician/user or may be defined based on a type of the negative pressure wound therapy required. In some examples, the controller 114 is further configured to store the rate of increase RI1 of the negative wound pressure NP with respect to time T in the memory 118. It should be understood that the rate of increase RI1 of the negative wound pressure NP may be suitably adjusted based on application requirements. The rise line 128 is shown dotted for illustrative purposes as the rise line 128 represents a portion of the cycle 121 that is running in the background.

The fall line 126 of a subsequent cycle 121 of the plurality of cycles 121 extends from the rise line 128 of the cycle 121. In the illustrated example of FIG. 2, the fall line 126 of the cycle 121-2 extends from the rise line 128 of the cycle 121-1. Thus, the controller 114 determines the fall line 126 and the rise line 128 in each cycle 121 when the upper negative target pressure 122 and the lower negative target pressure 124 are received as the input.

In some examples, the controller 114 is further configured to operate the pump 110 prior to a first cycle 121-1 of the plurality of cycles 121 to increase the negative wound pressure NP from an ambient pressure 130 as shown by the line 132. The ambient pressure 130 is shown as 0 mm Hg in FIG. 2. Subsequently, the controller 114 may operate the pump 110 to generate the negative wound pressure NP based on the plurality of cycles 121. In each cycle 121, the controller 114 is further configured to switch off the pump 110 when the negative wound pressure NP reaches the upper negative target pressure 122 of the cycle 121. The negative wound pressure NP may then reduce due to the leakage 116 in the system 100 while the pump 110 is switched off. In the absence of any leakage in the system 100, the negative wound pressure NP may remain constant at the upper negative target pressure 122.

In each cycle 121, the controller 114 is further configured to switch on the pump 110 when the negative wound pressure NP decreases from the upper negative target pressure 122 to the rise line 128 of the cycle 121 above the lower negative target pressure 124 due to the leakage 116 from the wound site 102 after switching off the pump 110. In other words, the controller 114 switches on the pump 110 when the negative wound pressure NP decreases from the upper negative target pressure 122 to a value on the rise line 128, the value being greater than the lower negative target pressure 124. Therefore, the controller 114 switches on the pump 110 when the negative wound pressure NP intersects the rise line 128 above the lower negative target pressure 124. In each cycle 121, the controller 114 is further configured to operate the pump 110 such that the negative wound pressure NP increases along the rise line 128 of the cycle 121. Subsequently, the controller 114 is further configured to switch off the pump 110 when the negative wound pressure NP reaches the upper negative target pressure 122 of the cycle 121. The process is then continued for the subsequent cycle 121. As the leakage flow through the leakage 116 increases further, the variation in the negative wound pressure NP with respect to time T may be more aligned with the fall line 126 and the rise line 128.

Thus, the system 100 of the present disclosure may conserve energy by not allowing the negative wound pressure NP to unnecessarily reduce to the lower negative target pressure 124 and then driving the pump 110 from the lower negative target pressure 124 to the upper negative target pressure 122. Instead, the controller 114 may operate the pump 110 to increase the negative wound pressure NP when the negative wound pressure NP decreases to the rise line 128 of the cycle 121, thereby reducing a power required by the pump 110 to drive the negative wound pressure NP to the upper negative target pressure 122. In the illustrated embodiment of FIG. 2, the controller 114 may apply an average negative pressure of 75 mm Hg to the wound site 102. In case of no leakage, the average negative pressure would have been the upper negative target pressure 122. The variation in the negative wound pressure NP appears only when there is leakage (e.g., the leakage 116) in the system 100. The reduction in the average negative pressure may not significantly affect the therapeutic benefits of the negative pressure wound therapy while allowing an operational life of the batteries to be extended during the leakage condition L1.

In the present disclosure, the system 100 may allow the negative wound pressure NP to be increased to the upper negative target pressure 122 by momentarily running the pump 110 while not allowing the negative wound pressure NP to drop to the lower negative target pressure 124, thereby reducing the power required over time T for providing the negative pressure wound therapy. Thus, the controller 114 may control the application of the negative wound pressure NP at the wound site 102 in a manner that is not only therapeutically effective, but also allows for extending the operational life of batteries in case of the leakage 116 from the wound site 102.

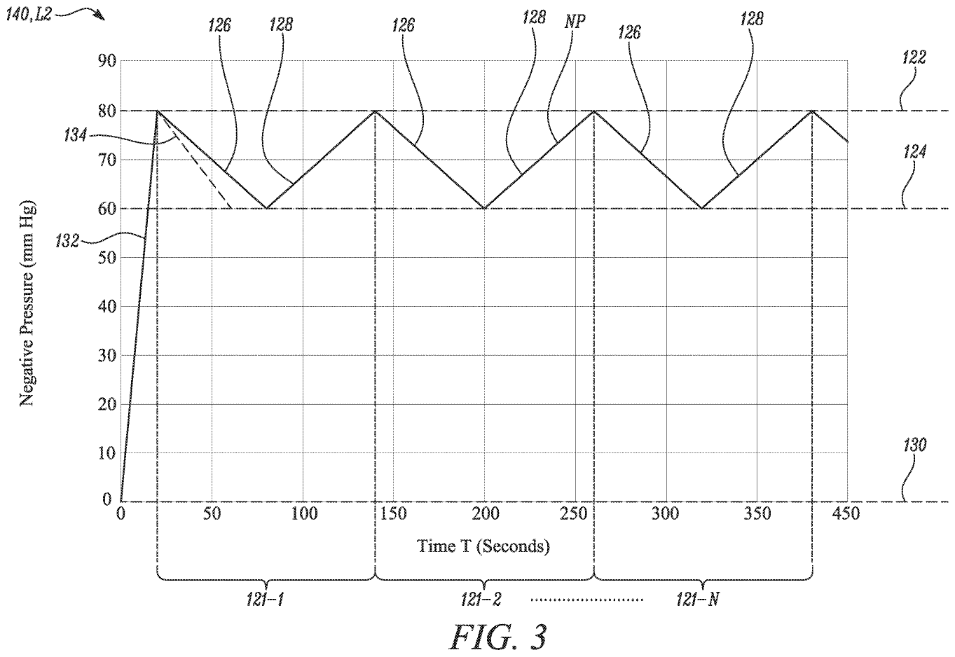

FIG. 3 is a graph 140 illustrating change in the negative wound pressure NP at the wound site 102 (shown in FIG. 1) with respect to time T. The graph 140 may represent a similar control strategy as that of the graph 120 (shown in FIG. 2). However, the graph 140 may correspond to a leakage condition L2 of the leakage 116 in the system 100. The leakage condition L2 may represent a leakage flow of 60 cc/min. Thus, the leakage flow during the leakage condition L2 is higher than the leakage flow during the leakage condition L1. However, it should be understood that the magnitude of the leakage flow during the leakage condition L2 is exemplary and may vary based on application requirements.

Referring to FIGS. 1 and 3, in some examples, in each cycle 121, the controller 114 is further configured to determine that the negative wound pressure NP crosses the fall line 126 of the cycle 121 due to the leakage 116 from the wound site 102 (e.g., the leakage condition L2) after the pump 110 is switched off. Specifically, a rate of decrease of the negative wound pressure NP due to the leakage 116 is greater than the rate of decrease RD1 or a slope of the fall line 126. Consequently, if the pump 110 is kept switched off, the negative wound pressure NP may fall below the lower negative target pressure 124 without intersecting the rise line 128. This is represented by the dashed line 134 for illustrative purposes.

In some examples, in each cycle 121, the controller 114 is further configured to operate the pump 110 to keep the negative wound pressure NP (as shown by the solid line) above the fall line 126 of the cycle 121 till the negative wound pressure NP reaches the rise line 128 of the cycle 121. Thus, during the leakage condition L2, the controller 114 may control the pump 110 to always maintain the negative wound pressure NP at the wound site 102 above the fall line 126 of the cycle 121. In such cases, the variation of the negative wound pressure NP with respect to time may be aligned with the fall line 126 and the rise line 128.

Thus, as the leakage flow through the leakage 116 increases, the controller 114 may control the pump 110 to keep the negative wound pressure NP close to the fall line 126 and the rise line 128 to maintain a controlled application of the negative wound pressure NP for therapeutic purposes. However, the average negative pressure applied at the wound site 102, i.e., 70 mm Hg in the illustrated example, may be reduced as compared to the average negative pressure at the wound site 102 in case of the leakage condition L1 (shown in FIG. 2).

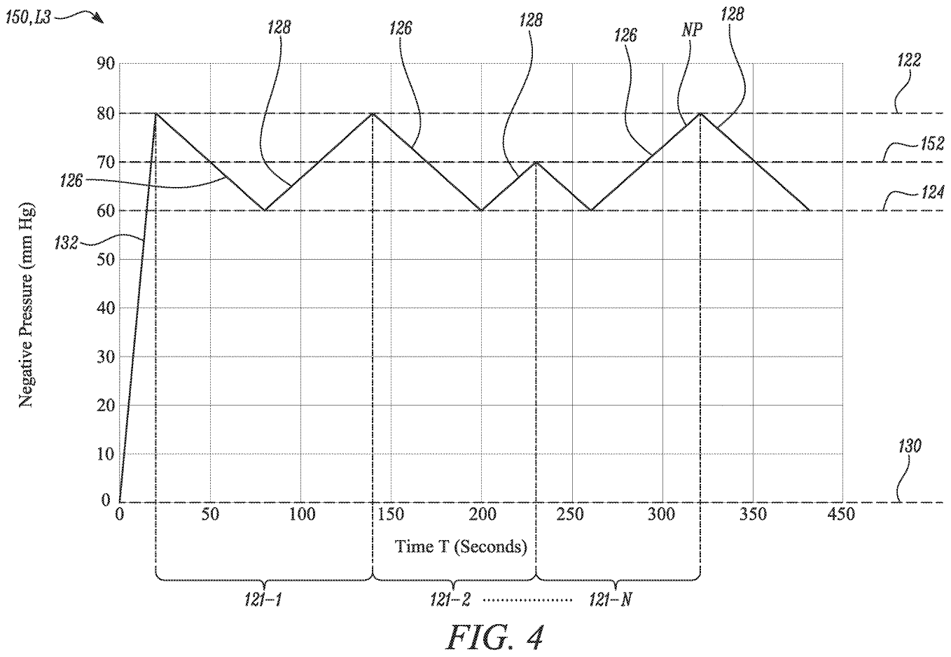

FIG. 4 is a graph 150 illustrating change in the negative wound pressure NP at the wound site 102 (shown in FIG. 1) with respect to time T. The graph 150 may represent a similar control strategy as that of the graph 120 (shown in FIG. 2). However, the graph 150 may correspond to a leakage condition L3 of the leakage 116 in the system 100. The leakage condition L3 may represent a leakage flow of 90 cc/min. Thus, the leakage flow during the leakage condition L3 is higher than the leakage flow during the leakage condition L2 (shown in FIG. 3) and the leakage condition L1 (shown in FIG. 2). Since the controller 114 may control the pump 110 to keep the negative wound pressure NP above the fall line 126 of each cycle 121, the negative wound pressure NP may be aligned with the fall line 126 as well as the rise line 128. In some examples, the fall line 126 and the rise line 128 corresponding to each cycle 121 may be determined by the controller 114 during start of each cycle 121.

In some examples, in each alternative cycle 121 of the plurality of cycles 121, the controller 114 is further configured to decrease the upper negative target pressure 122 relative to a preceding cycle 121 of the plurality of cycles 121 to an intermediate pressure value 152 between the upper negative target pressure 122 of the preceding cycle 121 and the lower negative target pressure 124 of the preceding cycle 121. In the illustrated embodiment of FIG. 4, in the cycle 121-2, the controller 114 is further configured to decrease the upper negative target pressure 122 relative to the cycle 121-1 to the intermediate pressure value 152 between the upper negative target pressure 122 of the cycle 121-1 and the lower negative target pressure 124 of the cycle 121-1. Thus, in the cycle 121-2, the negative wound pressure NP may rise to the intermediate pressure value 152 before the pump 110 is again switched off. This may reduce the average negative pressure delivered to the wound site 102 but also reduce the power requirements of the pump 110 over time at greater leakage flows, thereby extending the operational life of the batteries during such leakages.

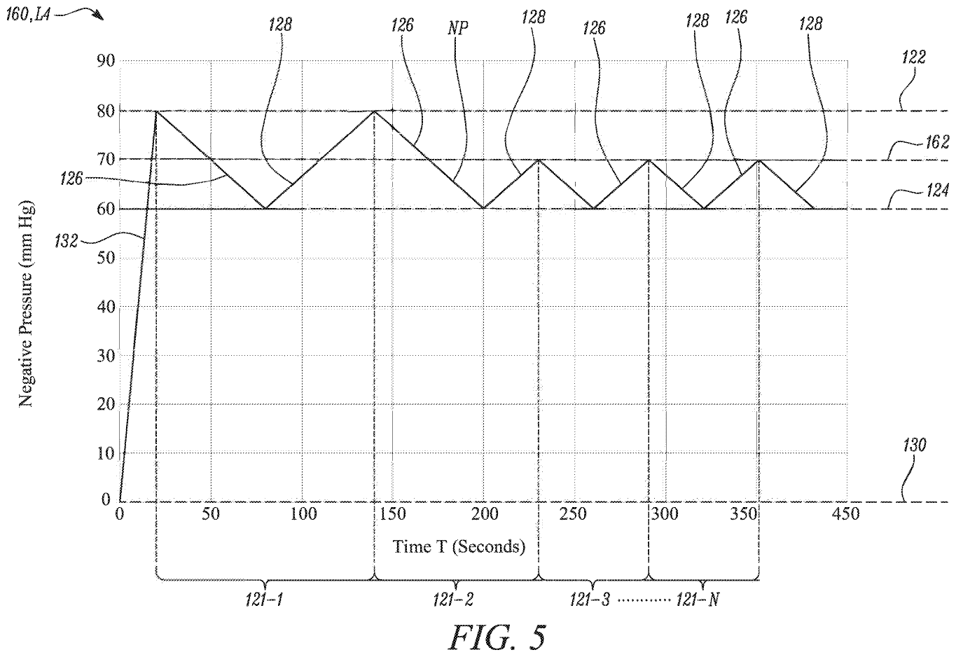

FIG. 5 is a graph 160 illustrating change in the negative wound pressure NP at the wound site 102 (shown in FIG. 1) with respect to time T. The graph 160 may represent a similar control strategy as that of the graph 120 (shown in FIG. 2). However, the graph 160 may correspond to a leakage condition L4 of the leakage 116 in the system 100. The leakage condition L4 may represent a leakage flow of 180 cc/min. Thus, the leakage flow during the leakage condition L4 is higher than the leakage flow during the leakage condition L3 (shown in FIG. 4), the leakage condition L2 (shown in FIG. 3), and the leakage condition L1 (shown in FIG. 2). Since the controller 114 may control the pump 110 to keep the negative wound pressure NP above the fall line 126 of each cycle 121, the negative wound pressure NP may be aligned with the fall line 126 as well as the rise line 128. In some examples, the fall line 126 and the rise line 128 corresponding to each cycle 121 may be determined by the controller 114 during start of each cycle 121.

In some examples, in each subsequent cycle 121 of the plurality of cycles 121, the controller 114 is further configured to decrease the upper negative target pressure 122 relative to a preceding cycle 121 of the plurality of cycles 121 to an intermediate pressure value 162 between the upper negative target pressure 122 of the preceding cycle 121 and the lower negative target pressure 124 of the preceding cycle 121. In the illustrated embodiment of FIG. 5, in the cycle 121-2, the controller 114 is further configured to decrease the upper negative target pressure 122 relative to the cycle 121-1 to the intermediate pressure value 162 between the upper negative target pressure 122 of the cycle 121-1 and the lower negative target pressure 124 of the cycle 121-1. Thus, in the subsequent cycles 121-2, . . . , 121-N, the negative wound pressure NP may rise to the intermediate pressure value 162 before the pump 110 is again switched off. This may reduce the average negative pressure delivered to the wound site 102 but also reduce the power requirements of the pump 110 over time at greater leakage flows, thereby extending the operational life of the batteries during such leakages.

It should be understood that the magnitude of the upper negative target pressure 122, the lower negative target pressure 124, the rate of decrease RD1, the rate of increase RI1, and the respective leakage flows corresponding to the leakage conditions L1, L2, L3, L4 as described above are exemplary and may vary based on application requirements.

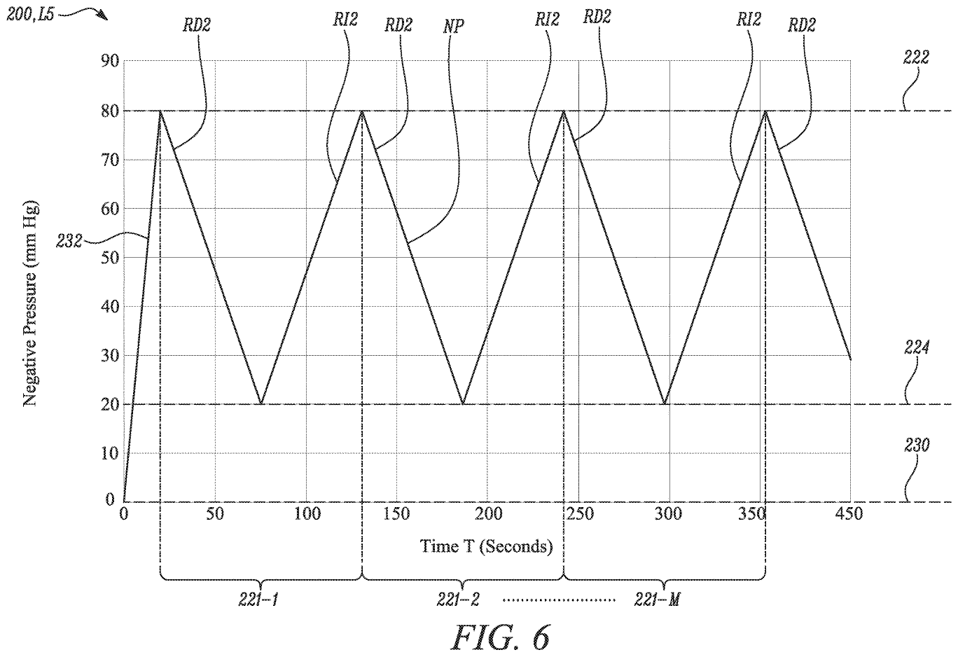

FIG. 6 is a graph 200 illustrating change in the negative wound pressure NP at the wound site 102 (shown in FIG. 1) with respect to time T, according to another embodiment of the present disclosure. Magnitude of the negative wound pressure NP (in mm Hg) is shown along the vertical axis or ordinate of the graph 200 and magnitude of time T (in seconds) is shown along the horizontal axis or abscissa.

Referring to FIGS. 1 and 6, the controller 114 is further configured to operate the pump 110 in a plurality of cycles 221-1, 221-2, . . . 221-M (collectively, cycles 221) based at least on the negative wound pressure NP, wherein M is a positive integer corresponding to a total number of cycles 221 (e.g., M=10, 20, etc.). In some examples, the plurality of cycles 221 are disposed adjacent to each other with respect to time T. In some examples, the graph 200 may correspond to a leakage condition L5 of the leakage 116 in the system 100. In some examples, the leakage condition L5 may represent a leakage flow of 30 cc/min. However, it should be understood that the magnitude of the leakage flow is purely exemplary, and the actual value may vary based on application.

In each cycle 221, the controller 114 is further configured to define an upper negative target pressure 222 and a lower negative target pressure 224. A magnitude of the upper negative target pressure 222 is greater than a magnitude of the lower negative target pressure 224. In some examples, the upper negative target pressure 222 and the lower negative target pressure 224 may be provided as an input to the controller 114 by the clinician/user. Alternatively, only the upper negative target pressure 222 is received as the input and the lower negative target pressure 224 may be preset and stored within the memory 118.

In the illustrated example of FIG. 6, the upper negative target pressure 222 is 80 mm Hg and the lower negative target pressure 224 is 20 mm Hg. However, it should be understood that the upper negative target pressure 222 and the lower negative target pressure 224 may assume any values based on application requirements. In the illustrated embodiment of FIG. 6, the upper negative target pressure 222 is constant across the plurality of cycles 221. Further, the lower negative target pressure 224 is constant across the plurality of cycles 221.

In each cycle 221, the controller 114 is further configured to operate the pump 110 prior to a first cycle 221-1 of the plurality of cycles 221 to increase the negative wound pressure NP from an ambient pressure 230 as shown by the line 232. The ambient pressure 230 is shown as 0 mm Hg in FIG. 6. Subsequently, the controller 114 may operate the pump 110 to generate the negative wound pressure NP based on the plurality of cycles 221. In each cycle 221, the controller 114 is further configured to switch off the pump 110 when the negative wound pressure NP reaches the upper negative target pressure 222 of the cycle 221. The negative wound pressure NP may then reduce due to the leakage 116 in the system 100 while the pump 110 is switched off. In the absence of any leakage in the system 100, the negative wound pressure NP may remain constant at the upper negative target pressure 222.

In each cycle 221, the controller 114 is further configured to determine a rate of decrease RD2 of the negative wound pressure NP with respect to time T from the upper negative target pressure 222 to the lower negative target pressure 224 of the cycle 221 due to the leakage 116 from the wound site 102 after switching off the pump 110. In some examples, the rate of decrease RD2 of the negative wound pressure NP may be determined based on the pressure signal S received from the pressure sensor 112. For example, the controller 114 may calculate a change in the negative wound pressure NP as received from the pressure sensor 112 with respect to time T to determine the rate of decrease RD2 of the negative wound pressure NP.

In each cycle 221, the controller 114 is further configured to determine a rate of increase RI2 of the negative wound pressure NP with respect to time T having a same magnitude as that of the rate of decrease RD2 of the negative wound pressure NP. In each cycle 221, the controller 114 is further configured to switch on the pump 110 when the negative wound pressure NP reaches the lower negative target pressure 224 of the cycle 221. In each cycle 221, the controller 114 is further configured to operate the pump 110 to increase the negative wound pressure NP from the lower negative target pressure 224 at the rate of increase RI2 of the negative wound pressure NP.

Thus, the controller 114 may match a time duration of decrease in the negative wound pressure NP with a time duration of increase in the negative wound pressure NP at the wound site 102 to maintain a controlled application of the negative wound pressure NP at the wound site 102. In some examples, as the leakage flow increases, the time duration of decrease in the negative wound pressure NP may reduce as well as the time duration of increase in the negative wound pressure NP, thereby increasing the number of cycles 221 in a given time duration.

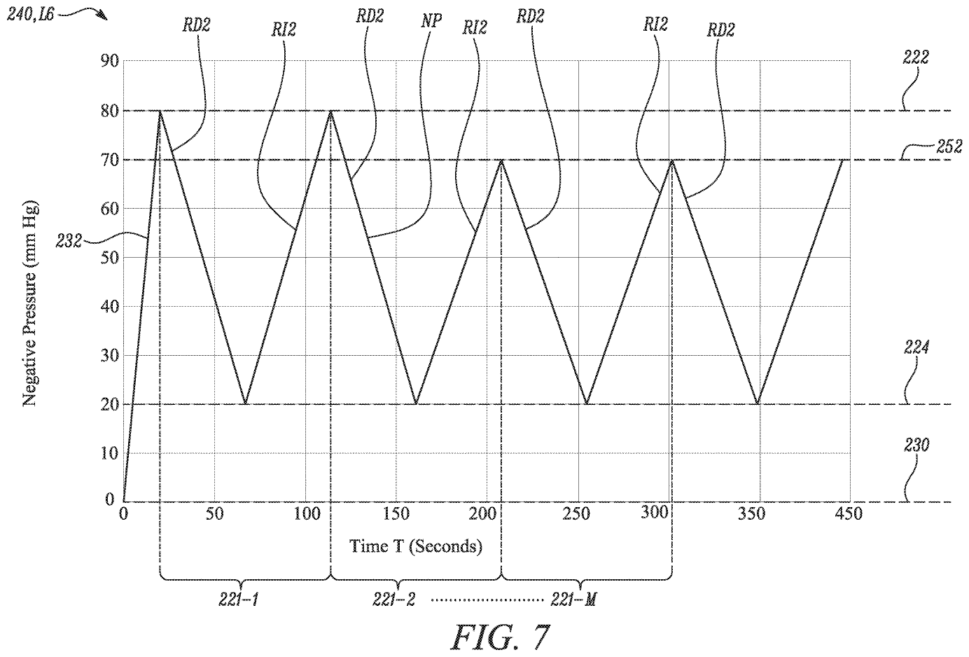

FIG. 7 is a graph 240 illustrating change in the negative wound pressure NP at the wound site 102 (shown in FIG. 1) with respect to time T. The graph 240 may represent a similar control strategy as that of the graph 200 (shown in FIG. 6). However, the graph 240 may correspond to a leakage condition L6 of the leakage 116 in the system 100. The leakage condition L6 may represent a leakage flow of 120 cc/min. Thus, the leakage flow during the leakage condition L6 is higher than the leakage flow during the leakage condition L5 (shown in FIG. 6). However, it should be understood that the magnitude of the leakage flow during the leakage condition L6 is exemplary and may vary based on application requirements.

In some examples, in each subsequent cycle 221 of the plurality of cycles 221, the controller 114 is further configured to decrease the upper negative target pressure 222 relative to a preceding cycle 221 of the plurality of cycles 221 to an intermediate pressure value 252 between the upper negative target pressure 222 of the preceding cycle 221 and the lower negative target pressure 224 of the preceding cycle 221. In the illustrated embodiment of FIG. 7, in each subsequent cycle 221-2, . . . 221-M, the controller 114 is further configured to decrease the upper negative target pressure 222 relative to the cycle 221-1 to the intermediate pressure value 252 between the upper negative target pressure 222 of the cycle 221-1 and the lower negative target pressure 224 of the cycle 221-1. Thus, in the subsequent cycles 221-2, . . . , 221-M, the negative wound pressure NP may rise to the intermediate pressure value 252 before the pump 110 is again switched off. This may reduce the average negative pressure delivered to the wound site 102 but also reduce the power requirements of the pump 110 over time at greater leakage flows, thereby extending the operational life of the batteries during such leakages.

It should be understood that the magnitude of the upper negative target pressure 222, the lower negative target pressure 224, and the respective leakage flows corresponding to the leakage conditions L5, L6 as described above are exemplary and may vary based on application requirements.

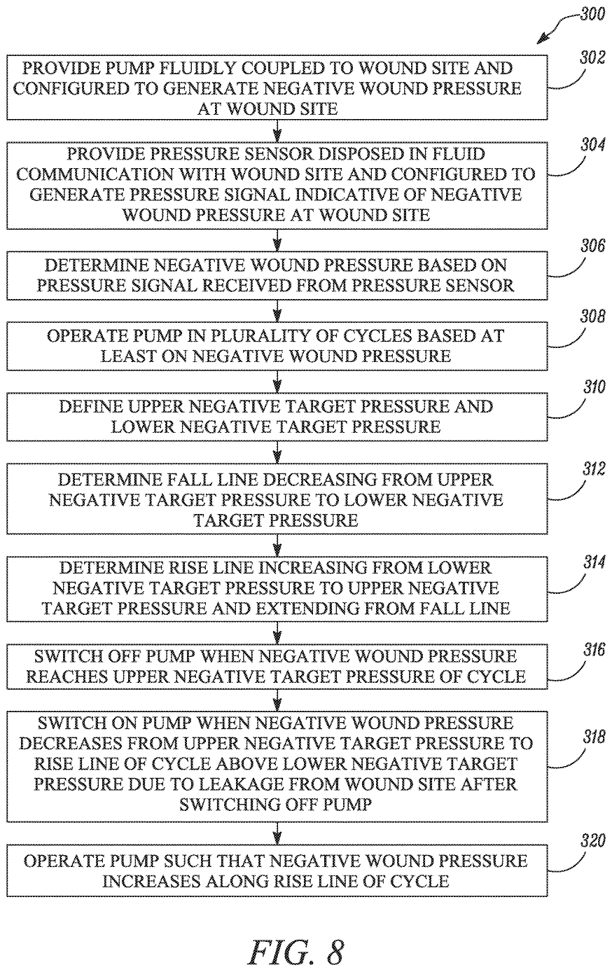

FIG. 8 is a flow chart illustrating a method 300 for negative pressure wound therapy. The method 300 will be described with reference to the system 100 of FIG. 1 and the graphs 120, 140, 150, 160 of FIGS. 2-5.

At step 302, the method 300 includes providing the pump 110 fluidly coupled to the wound site 102 and configured to generate the negative wound pressure NP at the wound site 102. At step 304, the method 300 further includes providing the pressure sensor 112 disposed in fluid communication with the wound site 102 and configured to generate the pressure signal S indicative of the negative wound pressure NP at the wound site 102.

At step 306, the method 300 further includes determining, via the controller 114, the negative wound pressure NP based on the pressure signal S received from the pressure sensor 112. At step 308, the method 300 further includes operating, via the controller 114, the pump 110 in the plurality of cycles 121 based at least on the negative wound pressure NP. The plurality of cycles 121 are disposed adjacent to each other with respect to time T.

At step 310, the method 300 further includes, in each cycle 121, defining the upper negative target pressure 122 and the lower negative target pressure 124. The magnitude of the upper negative target pressure 122 is greater than the magnitude of the lower negative target pressure 124. In some examples, the upper negative target pressure 122 is constant across the plurality of cycles 121. Further, the lower negative target pressure 124 is constant across the plurality of cycles 121.

At step 312, the method 300 further includes, in each cycle 121, determining the fall line 126 decreasing from the upper negative target pressure 122 to the lower negative target pressure 124. In some examples, determining the fall line 126 further includes defining the rate of decrease RD1 of the negative wound pressure NP with respect to time T from the upper negative target pressure 122 to the lower negative target pressure 124. In some examples, determining the fall line 126 further includes determining the fall line 126 based on the rate of decrease RD1 of the negative wound pressure NP, such that the fall line 126 is linear.

At step 314, the method 300 further includes, in each cycle 121, determining the rise line 128 increasing from the lower negative target pressure 124 to the upper negative target pressure 122 and extending from the fall line 126. The fall line 126 of the subsequent cycle 121 of the plurality of cycles 121 extends from the rise line 128 of the cycle 121. In some examples, determining the rise line 128 further includes defining the rate of increase RI1 of the negative wound pressure NP with respect to time T from the lower negative target pressure 124 to the upper negative target pressure 122. In some examples, determining the rise line 128 further includes determining the rise line 128 based on the rate of increase RI1 of the negative wound pressure NP, such that the rise line 128 is linear.

In some examples, the method 300 further includes operating, via the controller 114, the pump 110 prior to the first cycle 121-1 of the plurality of cycles 121 to increase the negative wound pressure NP from the ambient pressure 130.

At step 316, the method 300 further includes, in each cycle 121, switching off the pump 110 when the negative wound pressure NP reaches the upper negative target pressure 122 of the cycle 121.

At step 318, the method 300 further includes, in each cycle 121, switching on the pump 110 when the negative wound pressure NP decreases from the upper negative target pressure 122 to the rise line 128 of the cycle 121 above the lower negative target pressure 124 due to the leakage 116 from the wound site 102 after switching off the pump 110. At step 320, the method 300 further includes, in each cycle 121, operating the pump 110 such that the negative wound pressure NP increases along the rise line 128 of the cycle 121.

In some examples, the method 300 further includes, in each cycle 121, determining that the negative wound pressure NP crosses the fall line 126 of the cycle 121 due to the leakage 116 from the wound site 102 after the pump 110 is switched off. In some examples, the method 300 further includes, in each cycle 121, operating the pump 110 to keep the negative wound pressure NP above the fall line 126 of the cycle 121 till the negative wound pressure NP reaches the rise line 128 of the cycle 121.

In some examples, the method 300 further includes, in each alternative cycle 121 of the plurality of cycles 121, decreasing the upper negative target pressure 122 relative to the preceding cycle 121 of the plurality of cycles 121 to the intermediate pressure value 152 between the upper negative target pressure 122 of the preceding cycle 121 and the lower negative target pressure 124 of the preceding cycle 121.

In some examples, the method 300 further includes, in each subsequent cycle 121 of the plurality of cycles 121, decreasing the upper negative target pressure 122 relative to the preceding cycle 121 of the plurality of cycles 121 to the intermediate pressure value 152 between the upper negative target pressure 122 of the preceding cycle 121 and the lower negative target pressure 124 of the preceding cycle 121.

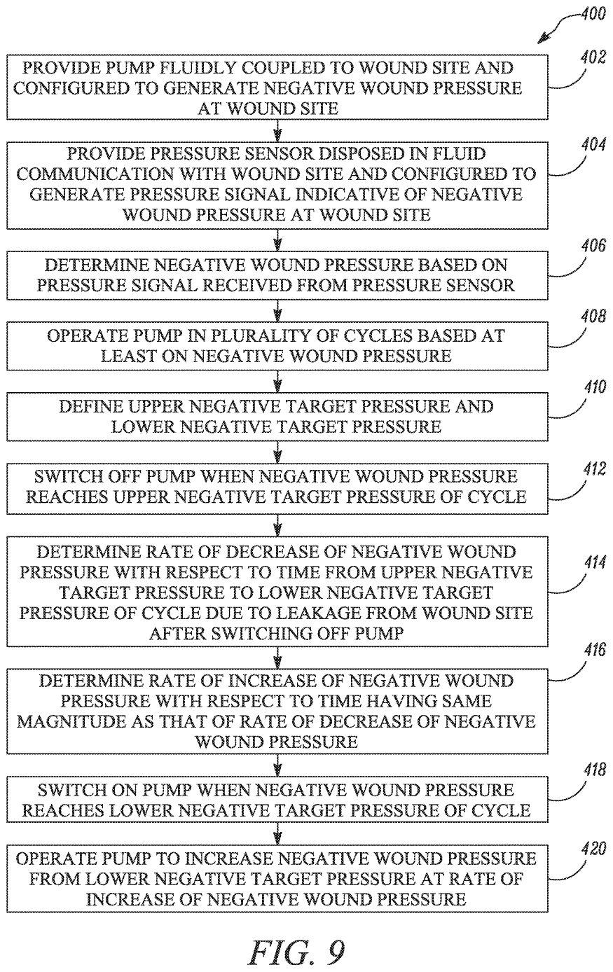

FIG. 9 is a flow chart illustrating a method 400 for negative pressure wound therapy. The method 400 will be described with reference to the system 100 of FIG. 1 and the graphs 200, 250 of FIGS. 6-7.

At step 402, the method 400 includes providing the pump 110 fluidly coupled to the wound site 102 and configured to generate the negative wound pressure NP at the wound site 102. At step 404, the method 400 further includes providing the pressure sensor 112 disposed in fluid communication with the wound site 102 and configured to generate the pressure signal S indicative of the negative wound pressure NP at the wound site 102.

At step 406, the method 400 further includes determining, via the controller 114, the negative wound pressure NP based on the pressure signal S received from the pressure sensor 112. At step 408, the method 300 further includes operating, via the controller 114, the pump 110 in the plurality of cycles 221 based at least on the negative wound pressure NP. The plurality of cycles 221 are disposed adjacent to each other with respect to time T.

At step 410, the method 400 further includes, in each cycle 221, defining the upper negative target pressure 222 and the lower negative target pressure 224. The magnitude of the upper negative target pressure 222 is greater than the magnitude of the lower negative target pressure 224. In some examples, the upper negative target pressure 222 is constant across the plurality of cycles 221. Further, the lower negative target pressure 224 is constant across the plurality of cycles 221.

In some examples, the method 400 further includes, in each cycle 221, operating, via the controller 114, the pump 110 prior to the first cycle 221-1 of the plurality of cycles 221 to increase the negative wound pressure NP from the ambient pressure 230.

At step 412, the method 400 further includes, in each cycle 221, switching off the pump 110 when the negative wound pressure NP reaches the upper negative target pressure 222 of the cycle 221.

At step 414, the method 400 further includes, in each cycle 221, determining the rate of decrease RD2 of the negative wound pressure NP with respect to time T from the upper negative target pressure 222 to the lower negative target pressure 224 of the cycle 221 due to the leakage 116 from the wound site 102 after switching off the pump 110. At step 416, the method 400 further includes, in each cycle 221, determining the rate of increase RI2 of the negative wound pressure NP with respect to time T having the same magnitude as that of the rate of decrease RD2 of the negative wound pressure NP.

At step 418, the method 400 further includes, in each cycle 221, switching on the pump 110 when the negative wound pressure NP reaches the lower negative target pressure 224 of the cycle 221. At step 420, the method 400 further includes, in each cycle 221, operating the pump 110 to increase the negative wound pressure NP from the lower negative target pressure 224 at the rate of increase RI2 of the negative wound pressure NP.

In some examples, the method 400 further includes, in each subsequent cycle 221 of the plurality of cycles 221, decreasing the upper negative target pressure 222 relative to the preceding cycle 221 of the plurality of cycles 221 to the intermediate pressure value 252 between the upper negative target pressure 222 of the preceding cycle 221 and the lower negative target pressure 224 of the preceding cycle 221.

The system 100 of the present disclosure may control the application of the negative wound pressure NP at the wound site 102 in a manner that is not only therapeutically effective, but also allows for extending an operational life of batteries in case of the leakage 116 from the wound site 102. The average negative pressure applied at the wound site 102 may not be significantly reduced due to the proposed control strategy, thereby maintaining the desired therapeutic effects of the negative wound pressure NP at the wound site 102. The system 100 may be advantageously applied to small portable therapy devices that typically run on batteries.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A system for negative pressure wound therapy, the system comprising:
a pump fluidly coupled to a wound site and configured to generate a negative wound pressure at the wound site;
a pressure sensor disposed in fluid communication with the wound site and configured to generate a pressure signal indicative of the negative wound pressure at the wound site; and
a controller communicably coupled to the pump and the pressure sensor, wherein the controller is configured to determine the negative wound pressure based on the pressure signal received from the pressure sensor and operate the pump in a plurality of cycles based at least on the negative wound pressure, wherein the plurality of cycles are disposed adjacent to each other with respect to time, and wherein, in each cycle, the controller is further configured to:
define an upper negative target pressure and a lower negative target pressure, wherein a magnitude of the upper negative target pressure is greater than a magnitude of the lower negative target pressure;
determine a fall line decreasing from the upper negative target pressure to the lower negative target pressure;
determine a rise line increasing from the lower negative target pressure to the upper negative target pressure and extending from the fall line, wherein the fall line of a subsequent cycle of the plurality of cycles extends from the rise line of the cycle;
switch off the pump when the negative wound pressure reaches the upper negative target pressure of the cycle;
switch on the pump when the negative wound pressure decreases from the upper negative target pressure to the rise line of the cycle above the lower negative target pressure due to a leakage from the wound site after switching off the pump; and
operate the pump such that the negative wound pressure increases along the rise line of the cycle.

2. The system of claim 1, wherein the controller is further configured to operate the pump prior to a first cycle of the plurality of cycles to increase the negative wound pressure from an ambient pressure.

3. The system of claim 1, wherein, in each cycle, the controller is further configured to:
define a rate of decrease of the negative wound pressure with respect to time from the upper negative target pressure to the lower negative target pressure;
and determine the fall line based on the rate of decrease of the negative wound pressure, such that the fall line is linear.

4. The system of claim 1, wherein, in each cycle, the controller is further configured to:
define a rate of increase of the negative wound pressure with respect to time from the lower negative target pressure to the upper negative target pressure; and
determine the rise line based on the rate of increase of the negative wound pressure, such that the rise line is linear.

5. The system of claim 1, wherein, in each alternative cycle of the plurality of cycles, the controller is further configured to decrease the upper negative target pressure relative to a preceding cycle of the plurality of cycles to an intermediate pressure value between the upper negative target pressure of the preceding cycle and the lower negative target pressure of the preceding cycle.

6. The system of claim 1, wherein, in each subsequent cycle of the plurality of cycles, the controller is further configured to decrease the upper negative target pressure relative to a preceding cycle of the plurality of cycles to an intermediate pressure value between the upper negative target pressure of the preceding cycle and the lower negative target pressure of the preceding cycle.

7. The system of claim 1, wherein the upper negative target pressure is constant across the plurality of cycles.

8. The system of claim 1, wherein the lower negative target pressure is constant across the plurality of cycles.

9. The system of claim 1, wherein, in each cycle, the controller is further configured to:
determine that the negative wound pressure crosses the fall line of the cycle due to the leakage from the wound site after the pump is switched off; and
operate the pump to keep the negative wound pressure above the fall line of the cycle till the negative wound pressure reaches the rise line of the cycle.

10. The system of claim 1, further comprising a wound dressing covering the wound site and defining a sealed volume around the wound site, wherein the pump is configured to generate the negative wound pressure within the sealed volume, wherein the pressure sensor is in fluid communication with the sealed volume, and wherein the leakage occurs from the sealed volume.

11. A system for negative pressure wound therapy, the system comprising:
a pump fluidly coupled to a wound site and configured to generate a negative wound pressure at the wound site;
a pressure sensor disposed in fluid communication with the wound site and configured to generate a pressure signal indicative of the negative wound pressure at the wound site; and
a controller communicably coupled to the pump and the pressure sensor, wherein the controller is configured to determine the negative wound pressure based on the pressure signal received from the pressure sensor and operate the pump in a plurality of cycles, wherein the plurality of cycles are disposed adjacent to each other with respect to time, and wherein, in each cycle, the controller is further configured to:
define an upper negative target pressure and a lower negative target pressure, wherein a magnitude of the upper negative target pressure is greater than a magnitude of the lower negative target pressure;
switch off the pump when the negative wound pressure reaches the upper negative target pressure of the cycle;
determine a rate of decrease of the negative wound pressure with respect to time from the upper negative target pressure to the lower negative target pressure of the cycle due to a leakage from the wound site after switching off the pump;
determine a rate of increase of the negative wound pressure with respect to time having a same magnitude as that of the rate of decrease of the negative wound pressure;
switch on the pump when the negative wound pressure reaches the lower negative target pressure of the cycle; and
operate the pump to increase the negative wound pressure from the lower negative target pressure at the rate of increase of the negative wound pressure.

12.-16. (canceled)

17. A method for negative pressure wound therapy, the method comprising:
providing a pump fluidly coupled to a wound site and configured to generate a negative wound pressure at the wound site;
providing a pressure sensor disposed in fluid communication with the wound site and configured to generate a pressure signal indicative of the negative wound pressure at the wound site;
determining, via a controller, the negative wound pressure based on the pressure signal received from the pressure sensor; and
operating, via the controller, the pump in a plurality of cycles based at least on the negative wound pressure, wherein the plurality of cycles are disposed adjacent to each other with respect to time, and wherein each cycle comprises:
defining an upper negative target pressure and a lower negative target pressure, wherein a magnitude of the upper negative target pressure is greater than a magnitude of the lower negative target pressure;
determining a fall line decreasing from the upper negative target pressure to the lower negative target pressure;
determining a rise line increasing from the lower negative target pressure to the upper negative target pressure and extending from the fall line, wherein the fall line of a subsequent cycle of the plurality of cycles extends from the rise line of the cycle;
switching off the pump when the negative wound pressure reaches the upper negative target pressure of the cycle;
switching on the pump when the negative wound pressure decreases from the upper negative target pressure to the rise line of the cycle above the lower negative target pressure due to a leakage from the wound site after switching off the pump; and
operating the pump such that the negative wound pressure increases along the rise line of the cycle.

18. The method of claim 17, further comprising operating, via the controller, the pump prior to a first cycle of the plurality of cycles to increase the negative wound pressure from an ambient pressure.

19. The method of claim 17, wherein determining the fall line further comprises:
defining a rate of decrease of the negative wound pressure with respect to time from the upper negative target pressure to the lower negative target pressure; and
determining the fall line based on the rate of decrease of the negative wound pressure, such that the fall line is linear.

20. The method of claim 17, wherein determining the rise line further comprises:
defining a rate of increase of the negative wound pressure with respect to time from the lower negative target pressure to the upper negative target pressure; and
determining the rise line based on the rate of increase of the negative wound pressure, such that the rise line is linear.

21. The method of claim 17, further comprising, in each alternative cycle of the plurality of cycles, decreasing the upper negative target pressure relative to a preceding cycle of the plurality of cycles to an intermediate pressure value between the upper negative target pressure of the preceding cycle and the lower negative target pressure of the preceding cycle.

22. The method of claim 17, further comprising, in each subsequent cycle of the plurality of cycles, decreasing the upper negative target pressure relative to a preceding cycle of the plurality of cycles to an intermediate pressure value between the upper negative target pressure of the preceding cycle and the lower negative target pressure of the preceding cycle.

23. The method of claim 17, wherein the upper negative target pressure is constant across the plurality of cycles.

24. The method of claim 17, wherein the lower negative target pressure is constant across the plurality of cycles.

25. The method of claim 17, wherein each cycle further comprises:
determining that the negative wound pressure crosses the fall line of the cycle due to the leakage from the wound site after the pump is switched off; and
operating the pump to keep the negative wound pressure above the fall line of the cycle till the negative wound pressure reaches the rise line of the cycle.

26. A method for negative pressure wound therapy, the method comprising:
providing a pump fluidly coupled to a wound site and configured to generate a negative wound pressure at the wound site;
providing a pressure sensor disposed in fluid communication with the wound site and configured to generate a pressure signal indicative of the negative wound pressure at the wound site;
determining, via a controller, the negative wound pressure based on the pressure signal received from the pressure sensor; and
operating, via the controller, the pump in a plurality of cycles based at least on the negative wound pressure, wherein the plurality of cycles are disposed adjacent to each other with respect to time, and wherein each cycle comprises:
defining an upper negative target pressure and a lower negative target pressure, wherein a magnitude of the upper negative target pressure is greater than a magnitude of the lower negative target pressure;
switching off the pump when the negative wound pressure reaches the upper negative target pressure of the cycle;
determining a rate of decrease of the negative wound pressure with respect to time from the upper negative target pressure to the lower negative target pressure of the cycle due to a leakage from the wound site after switching off the pump;
determining a rate of increase of the negative wound pressure with respect to time having a same magnitude as that of the rate of decrease of the negative wound pressure;
switching on the pump when the negative wound pressure reaches the lower negative target pressure of the cycle; and
operating the pump to increase the negative wound pressure from the lower negative target pressure at the rate of increase of the negative wound pressure.

27.-30. (canceled)
